(19) United States
(12) Patent Application Publication
(10) Pub. No.: US 2016/0296725 A1
(43) Pub. Date: Oct. 13, 2016

(54) ANTI-SHOCK SYSTEM FOR PIC LINES AND OTHER CATHETERS

(71) Applicant: Wayne A. Calco, Santa Margarita, CA (US)

(72) Inventor: Wayne A. Calco, Santa Margarita, CA (US)

(21) Appl. No.: 15/094,765

(22) Filed: Apr. 8, 2016

Related U.S. Application Data
(60) Provisional application No. 62/145,718, filed on Apr. 10, 2015.

(51) Int. Cl. A61M 25/02; A61M 39/10

(52) U.S. Cl. CPC A61M 25/02; A61M 39/10; A61M 2025/0246; A61M 2025/026; A61M 2025/0213

(57) ABSTRACT

An anti-shock system for a catheter line inserted in a patient's body. The system includes a spring structure, a base and cover forming a housing with a catheter line entry port and exit port, the spring structure disposed within the housing. A first portion of a catheter line is positioned within the housing, with a distal end portion extending out the exit port, and a body entrance portion extending from the entry port. The spring structure is operatively coupled to a loop of the line and configured to absorb a pulling force exerted on the distal end by a spring action on the loop, without transmitting the pulling force directly to the body entrance portion of the catheter line and thereby prevent undesired migration of the catheter line within the patient's body.

### CROSS-REFERENCE TO RELATED APPLICATION

This application claims the benefit of U.S. Provisional Application No. 62/145,718 filed Apr. 10, 2015, the entire contents of which are hereby incorporated by reference.

### BACKGROUND

Catheters are used, for example, to deliver medications or devices into a patient's body, or remove fluids or matter from the body. Peripherally inserted central catheters (PICC or PIC lines) are a common example, and are used for intravenous access over a prolonged period of time. PIC lines and catheters can be used to deliver chemotherapy, antibiotics or other medicines or nutrients to a patient, or to allow fluids to exit the body.

A problem with catheters such as PIC lines is to maintain the position relative to the body, since the catheters are susceptible to inadvertent tugging or pulling which might tend to cause the line to migrate, i.e. to pull the line out of the body by some distance.

### BRIEF DESCRIPTION OF THE DRAWINGS

Features and advantages of the disclosure will readily be appreciated by persons skilled in the art from the following detailed description when read in conjunction with the drawing wherein:

FIG. 1 is a diagrammatic view illustrating a PIC line as emerging from an insertion point in a patient's arm, with an exemplary embodiment of an anti-shock system for the PIC line.

FIG. 2 is an exploded view illustrating features of an exemplary embodiment of an anti-shock system.

FIGS. 2A and 2B are views of the wheel structure of the embodiment of FIG. 2.

FIG. 3 is an isometric view of the system of FIG. 2 with the catheter line in assembled form and the system cover removed.

FIG. 4 is an isometric view of the system of FIG. 2 in assembled form.

FIG. 5 is an exploded, isometric view of an alternate embodiment of an anti-shock system for a catheter.

FIG. 6 is an isometric view of the system of FIG. 5 in assembled form.

FIG. 7 is an isometric view similar to that of FIG. 6, but with the cover removed.

FIG. 8 is an isometric view of a further embodiment of an anti-shock system for a catheter.

FIG. 9 is a view similar to FIG. 8, but with the top cover removed to show the wheel structure and arrangement of the tubing.

FIG. 10 is an isometric exploded view of the system of FIGS. 8 and 9.

### DETAILED DESCRIPTION

In the following detailed description and in the several figures of the drawing, like elements are identified with like reference numerals. The figures may not be to scale, and relative feature sizes may be exaggerated for illustrative purposes.

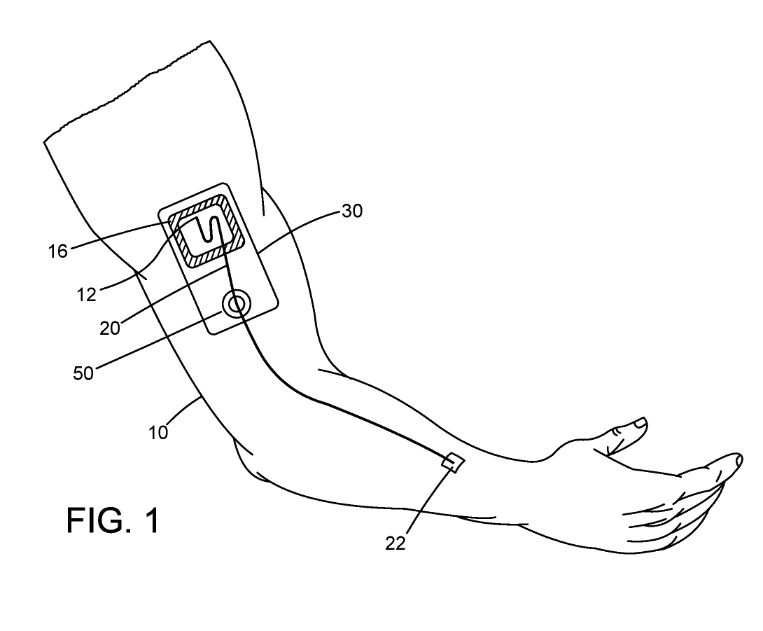

FIGS. 1-4 illustrate an exemplary embodiment of an anti-shock system 50 for a catheter such as a PIC line 20. The PIC line 20 is inserted into a patient's blood vessel at a body insertion point 12 in the patient's arm 10, in this example, and run into a blood vessel to terminate at a desire point in the patient's body. A fitting 22 at a distal end of the line 20 provides a connection to a device or system for delivering a therapy intravenously to the patient through the PIC line 20. The insertion point is typically covered by clear or translucent adhesive tape 16.

In the absence of the anti-shock system 50, it is apparent that the catheter 20 is susceptible to pulling or tugging on the line, typically at or adjacent the distal end. This can occur inadvertently, e.g. when the line is disconnected from the delivery system. Pulling on the catheter can cause the catheter to migrate, i.e. to move the catheter within the blood vessel, with the interior end moved away from a desired therapy point in the patient's body. Conventionally, stabilization devices have been employed to secure the catheter to the patient's skin using aggressive adhesive materials. These devices can be difficult to remove without damaging the patient's skin, and also can allow tugging on the line at its distal end from applying pressure at the insertion point, since the skin itself can move in relation to underlying body structures and the blood vessel in which the catheter is inserted.

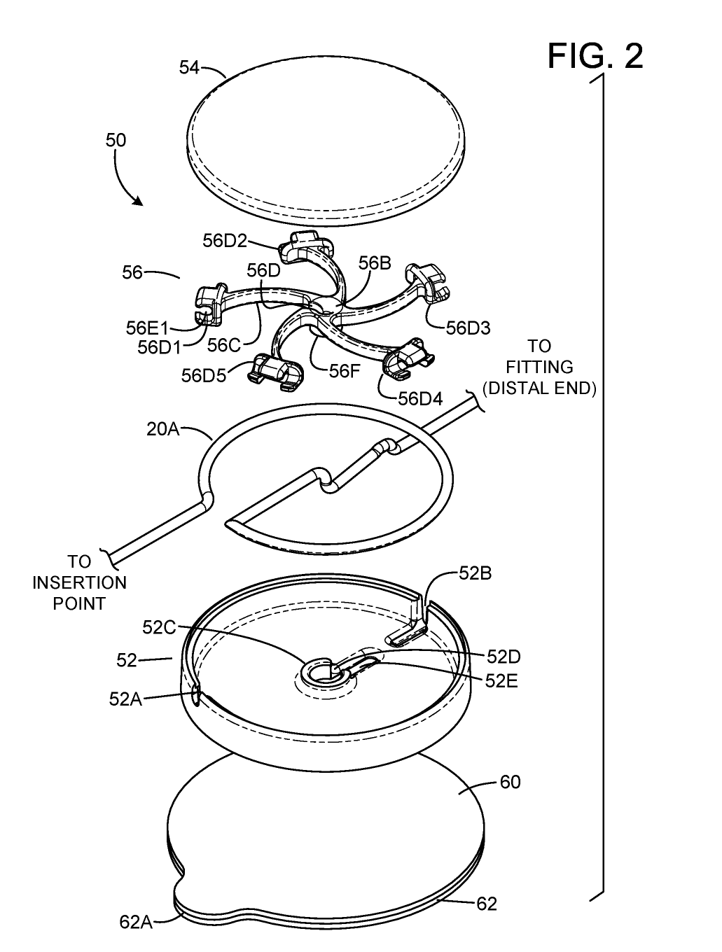

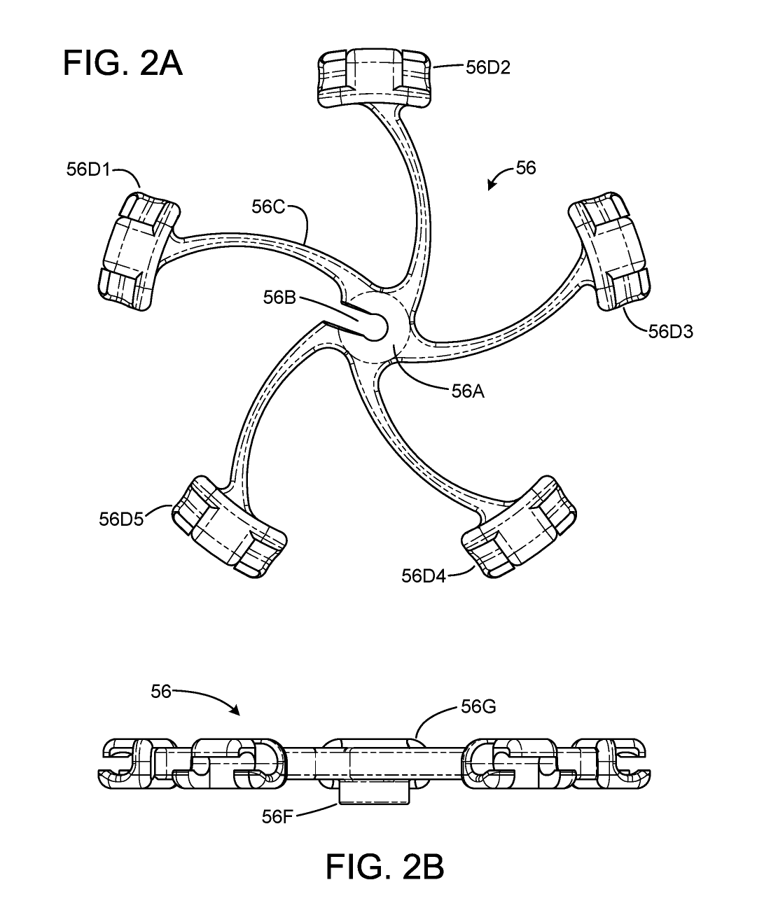

In accordance with aspects of the invention, the PIC line is connected, or travels through, an anti-shock system 50, more fully illustrated in FIGS. 2-4. The system 50 utilizes a spring action on a length of the catheter formed in a loop, so that the loop can absorb forces pulling on the distal end of the catheter without transmitting the pulling force directly to the insertion point.

The system 50 includes a housing formed by base 52 and a cover 54, which may be fabricated by injection molding of a plastic material such as ABS. The base 52 has opposed peripheral slots 52A, 52B which provide ports for entry and egress of the catheter line 20 from the system. The base has a central opening and boss 52C, with a slot 52D in the boss. A slot opening 52E is formed in the base and communicates with the central opening in the boss. The boss is configured to receive the lower portion 56F of the hub 56A of wheel structure 56, shown in further detail in FIGS. 2A and 2B. The slot opening 52E of the wheel structure is aligned with the slot 52D in the boss 52C.

The wheel structure is fabricated from a material having some flexibility, such as Nylon or Delrin, for example. The wheel structure includes the hub 56A which has open slot 56. The engagement of the protrusion 56A into boss 52C in this exemplary embodiment is an interference fit, which prevents the wheel structure from rotating within the housing. In other embodiments, the engagement may be by different structures, for example a protrusion from hub 52C fitting into a corresponding opening or recess in the boss 52C.

The wheel structure further includes curved or bowed spokes 56C extending outwardly from the hub, terminating in fitments which define line receptacles. In this embodiment, there are five curved spokes terminating in fitments 56D1-56D5, respectively, so that there are five receptacles 56E1-56E5. The wheel structure is a unitary structure in this exemplary embodiment.

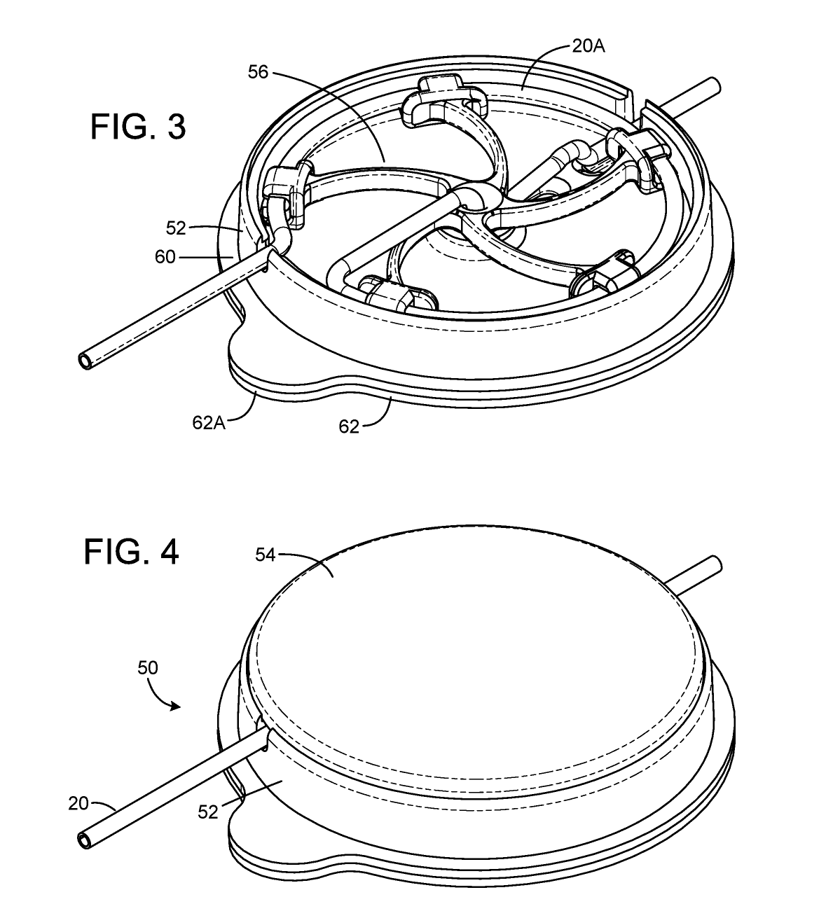

The PIC line 20 has a loop 20A formed in it, which is supported by the engagement of the line within the spoke receptacles 56E. In this example, the line 20 enters the housing through port 52A, and is connected in clockwise fashion to respective receptacles 56E1, 56E2, 56E3, 56E4, with the tag end turned 90 degrees and directed toward the center hub 56A. The line may be turned downwardly into the hub opening and then radially outwardly from the hub toward the exit port 52B, where it exits the housing. The configuration in which the flexible line is formed is depicted in FIG. 2 and in FIG. 3. The slot 52E provided some clearance room for the adjacent portion of the line.

The cover 54 is configured to fit onto the base 52, e.g. in an interference fit, with FIG. 4 illustrating the assembled condition. The hub 52C of the wheel structure is captured between the base and the cover, and prevented from moving or rotating.

In an exemplary embodiment, the receptacles at the distal ends of the curved spokes 56C are configured to allow the line 20 to snap into the receptacle, with some frictional engagement.

The system 50 further includes an adhesive layer 60 attached to the bottom surface of the base 52, with a release cover layer 62 over laying the adhesive layer. The user may use tab 62A to pull the release layer off the adhesive layer prior to application of the system to the patient's arm or other site.

Now consider the situation in which a patient experiences a pulling or tugging on the distal end of the PIC line 20, typically close to the fitting 22 end. The pulling force is transmitted by the line 20 to the loop 20A and to the receptacles 56D5-56D1, in turn tending to bend the curved spokes, rather than being transmitted to the line 20 at the insertion site 12. The receptacles frictionally engage the line of the loop 20A, so that the line does not slip through the receptacles when the line is tugged. Instead of migrating the line 20 within the patient's body, the tugging force is taken up by the spring action of the wheel structure 56 within the housing, allowing some portion of the line within the housing to be pulled out of the housing structure if the force is high enough. Once the tugging force is removed, the spring action of the wheel structure 56 will retract the line back into the housing structure.

The portion of the catheter line 20 which extends toward the insertion point on the patient's body can be of sufficient length to serve as the portion of the catheter inserted into the patient's body. Alternatively, the portion may be terminated in a fitting, to allow connection to a separate catheter line end of the inserted catheter.

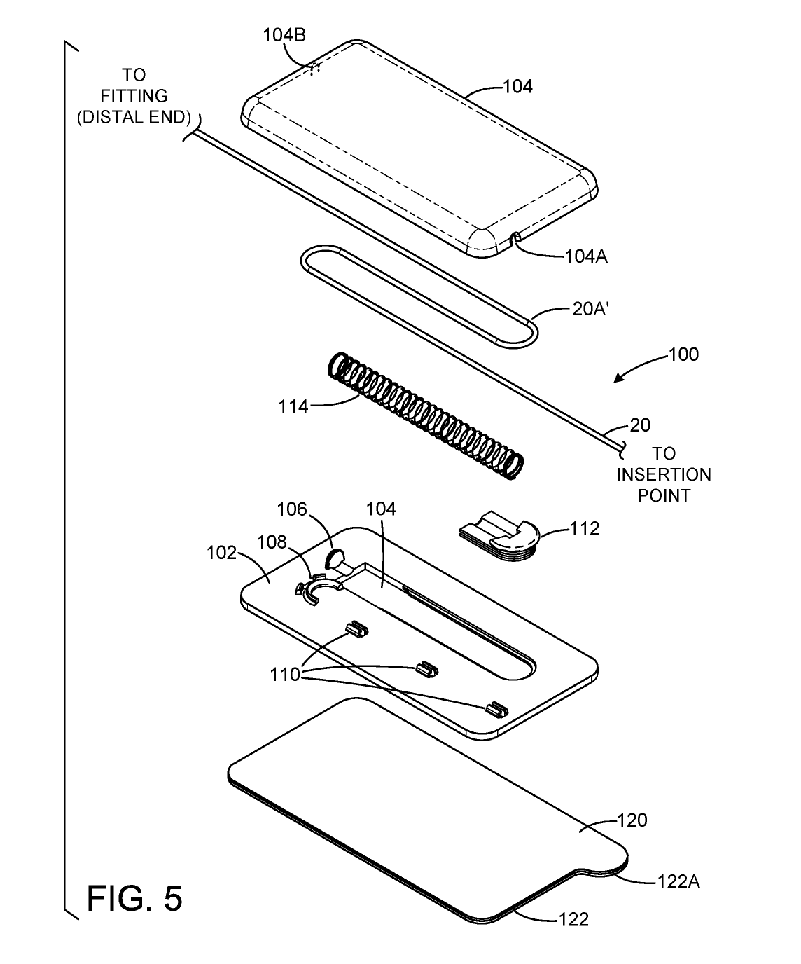

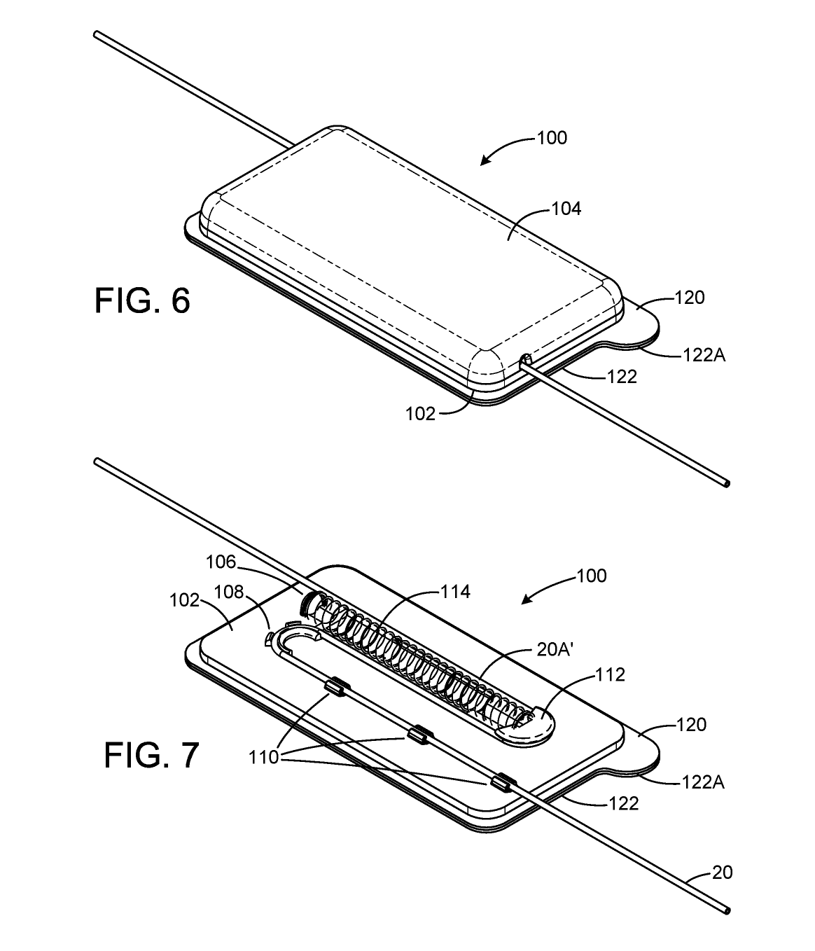

An alternate embodiment of an anti-shock system 100 is illustrated in FIGS. 5-7. This embodiment includes base 102 and cover 104, with adhesive layer 120 attached to the bottom exterior surface of the base to adhere the system to the patient's arm or other site. In this embodiment, a coil spring 114 takes the place of the wheel structure 56 of the embodiment of FIGS. 1-4. The spring is mounted between a stationary cleat 106 defined on base 102 and a slider 112 in turn mounted for sliding movement along slot 104 formed in base structure 102. The loop 20A′ formed in the catheter line 20 is fitted around the slider, a second fixed cleat 108, and through cleats 110. The line 20 is passed through entry port 104A defined in the cover 104, and through exit port 104B. The entry port 104A is configured to frictionally engage the line 20 to prevent it from slipping through the port as a pulling force is applied. Alternatively, another cleat can be provided just inside the base at the port 104 to engage the line and prevent its slippage. Also, the slider 112 may have a groove formed therein to receive the line and frictionally engage it, preventing slippage.

Consider the situation in which the fitment end of the line 20 is pulled or tugged inadvertently. The pulling force will be applied to the slider 112, drawing it toward the cleat 106 and tending to compress the spring 114, allowing the distal end to pay out somewhat in reaction to the pulling force without applying tugging force to the line at the insertion point. Once the tugging force is removed from the line, the spring pushes the slider 112 back to the rest position, withdrawing the portion of line paid out back into the housing structure.

The base 102, cover 104 and slider 112 may be fabricated as plastic structures, typically by injection molding. The spring 114 may be a metal coil spring. Alternatively, other spring types may be used.

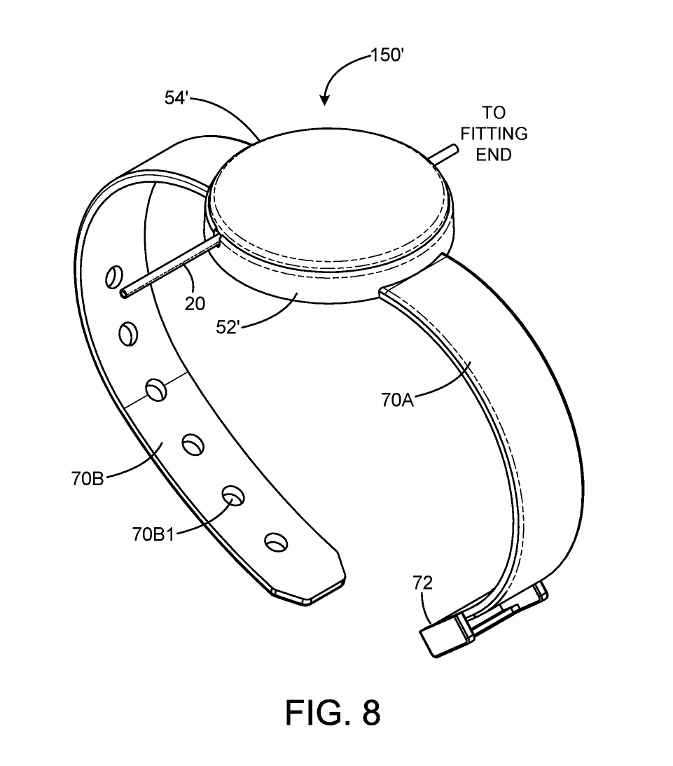

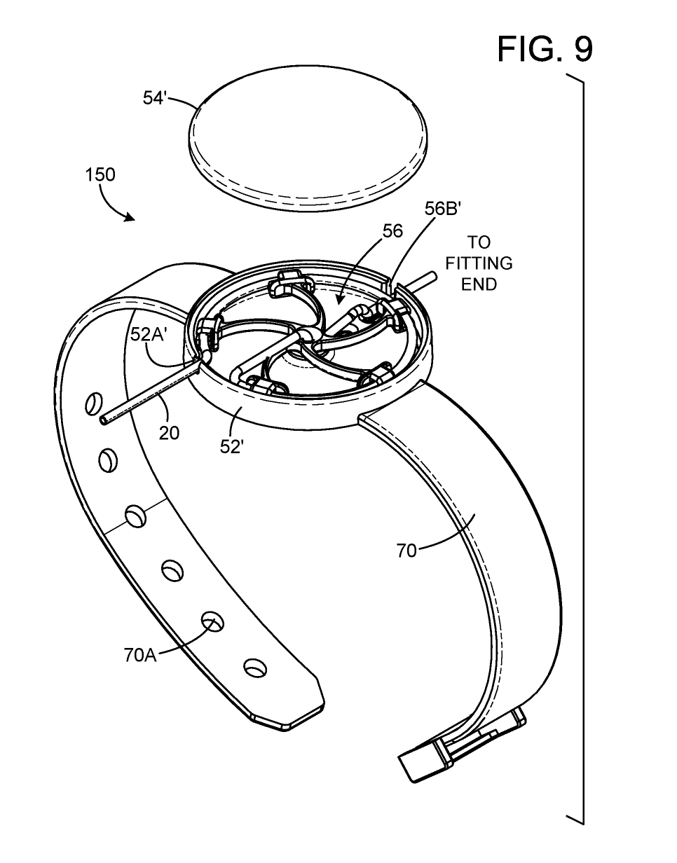

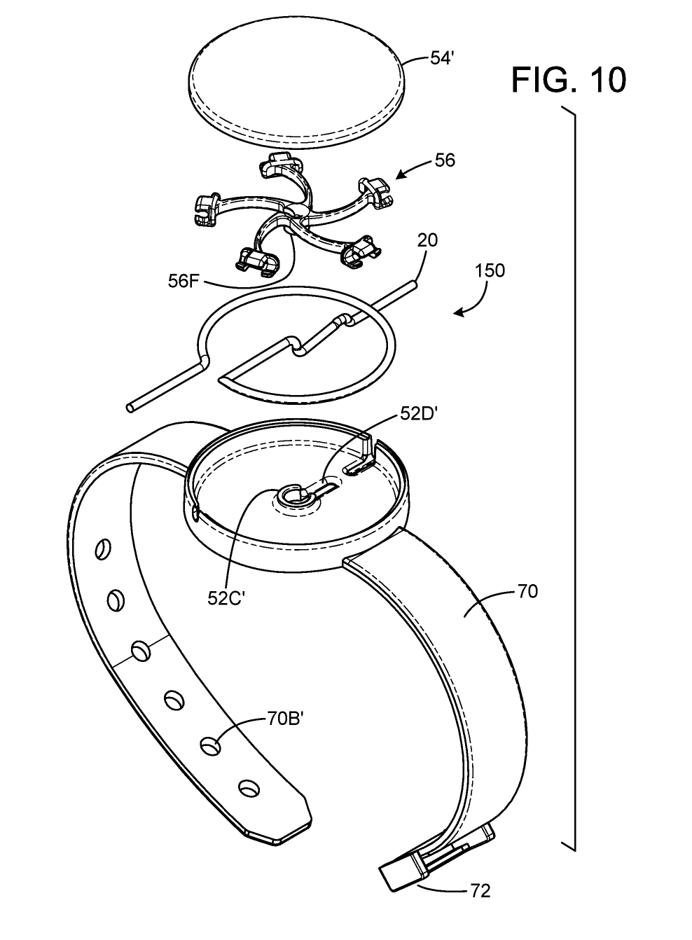

A further embodiment of an anti-shock system 150 is illustrated in FIGS. 8-10. This embodiment is similar to the embodiment of FIGS. 1-4; however, the base 52′ has straps 70A, 70B extending from opposite sides thereof, with a buckle or fastener 72 at the distal end of strap 70A. The system 150 may therefor omit the use of an adhesive layer to secure the system to the patient's body. Thus, the system 150′ includes a base 52′, a removable cover 54′, and a wheel 56 as in the embodiment 50 of FIGS. 1-4. The base 52′ includes a boss 52C′ with a slot 52D′ formed in the boss. The wheel hub 56F engages the boss as with the system 50. The catheter line 20 is engaged with the fitments formed in the wheel 56, as with the system 50, and is passed from inlet port 52A′, with a loop, and then back through the wheel hub and out the port 56B′ of the base 52′. As with the system 50, the wheel hub is prevented from rotation within the base, after the cover is attached. The hub 52C of the wheel structure is captured between the base and the cover, and prevented from moving or rotating.

The buckle 72 may use a pin (not shown) to enter one of the holes 70B1, a clasp, or even hoop-and-loop or other type of fastener, allowing the straps to secure the base 52′ to the patient's arm or wrist. This may avoid the use of an adhesive to secure the system 150 to the patient's arm or wrist, and make removal more convenient to the patient while avoiding the risk of injury to the patient's skin. The straps could be sized to attach to the patient's arm above the elbow, or to the patient's forearm, or to the patient's wrist. Alternatively, or in addition, the back of the base 52′ may include a less aggressive adhesive layer than is used with the system 50.

Once the system 150 has been attached to the patient's body by use of the straps, the system 150 protects against migration of the line 20 within the patient's body in the same manner as described above with respect to system 50.

Although the foregoing has been a description and illustration of specific embodiments of the subject matter, various modifications and changes thereto can be made by persons skilled in the art without departing from the scope and spirit of the invention.

What is claimed is:

1. An anti-shock system for a catheter line inserted in a patient's body, comprising:
a spring structure;
a base and cover forming a housing with a catheter line entry port and exit port, the spring structure disposed within the housing;
a first portion of a catheter line positioned within the housing, with a distal end portion of the catheter line extending out the exit port, and a body entrance portion of the catheter line extending from the entry port;
the spring operatively coupled to a loop of the line within the housing and configured to absorb a pulling force exerted on the distal end of the catheter line by a spring action on the loop, so that the loop can absorb forces pulling on the distal end portion of the catheter without transmitting the pulling force directly to the body entrance portion of the catheter line and thereby prevent undesired migration of the catheter line within the patient's body.

2. The system of claim 1, wherein the distal end portion of the catheter line is terminated in a fitting for removable attachment to a fluid reservoir.

3. The system of claim 1, further comprising:
an adhesive layer attached to a bottom exterior surface of the base for attaching the system to a patient's body.

4. The system of claim 1, wherein the spring structure comprises a wheel structure having a hub and a plurality of curved spokes extending outwardly from the hub, and a plurality of line receptacles at distal ends of the spokes for frictionally engaging the line loop, the wheel structure having sufficient flexibility to allow the loop to compress and allow some line within the housing to pay out toward the distal end portion in response to a tugging force applied to the distal end portion.

5. The system of claim 1, wherein the spring structure further comprises a slider member affixed to the loop and to a spring member, the spring structure biasing the slider member to a rest position, with the slider member slidable along a range of travel in response to a tugging force applied to the distal end portion.

6. The system of claim 5, wherein the spring member is a coil spring mounted between a first stationary cleat defined on the base and the slider member, and the loop is fitted around the slider, a second fixed cleat and a third fixed cleat.

7. The system of claim 1, further comprising:
first and second straps attached to the housing; and
a strap fastener for removably connecting distal ends of the first and second straps and securing the housing to the patient's body.

8. The system of claim 7, wherein the straps are sized to fit about and secure the system to a patient's wrist or arm.

9. An anti-shock system for a catheter line inserted in a patient's body, comprising:
a spring structure;
a base and cover forming a housing with a catheter line entry port and exit port, the spring structure disposed within the housing;
a first portion of a catheter line positioned within the housing, with a distal end portion of the catheter line extending out the exit port, and a body entrance portion of the catheter line extending from the entry port;
wherein the spring structure comprises a wheel structure having a hub and a plurality of curved spokes extending outwardly from the hub, and a plurality of line receptacles at distal ends of the spokes for frictionally engaging the line loop, the curved spokes of the wheel structure having sufficient flexibility to allow the loop to compress and allow some line within the housing to pay out toward the distal end portion in response to a tugging force applied to the distal end portion;
the spring operatively coupled to a loop of the line within the housing and configured to absorb a pulling force exerted on the distal end of the catheter line by a spring action on the loop, so that the loop can absorb forces pulling on the distal end portion of the catheter without transmitting the pulling force directly to the body entrance portion of the catheter line and thereby prevent undesired migration of the catheter line within the patient's body.

10. The system of claim 9, wherein the distal end portion of the catheter line is terminated in a fitting for removable attachment to a fluid reservoir.

11. The system of claim 9, wherein the hub is fitted into a boss structure of the base, and is not rotatable.

12. The system of claim 9, wherein the spokes of the wheel are configured such that, once the pulling force is removed, the spring action of the curved spokes retracts the portion back into the housing.

13. The system of claim 9, further comprising:
an adhesive layer attached to a bottom exterior surface of the base for securing the system to a patient's body.

14. The system of claim 9, further comprising:
first and second straps attached to the housing; and
a strap fastener for removably connecting distal ends of the first and second straps and securing the housing to the patient's body.

15. The system of claim 14, wherein the straps are sized to fit about and secure the system to a patient's wrist or arm.

16. An anti-shock system for a catheter line inserted in a patient's body, comprising:
a spring structure;
a base and cover forming a housing with a catheter line entry port and exit port, the spring structure disposed within the housing;
a first portion of a catheter line positioned within the housing, with a distal end portion of the catheter line extending out the exit port, and a body entrance portion of the catheter line extending from the entry port;
the spring operatively coupled to a loop of the line within the housing and configured to absorb a pulling force exerted on the distal end of the catheter line by a spring action on the loop, so that the loop can absorb forces pulling on the distal end portion of the catheter without transmitting the pulling force directly to the body entrance portion of the catheter line and thereby prevent undesired migration of the catheter line within the patient's body;
wherein the spring structure further comprises a slider member fitted to the loop and to a spring member, the spring structure biasing the slider member to a rest position, with the slider member slidable along a range of travel in response to a tugging force applied to the distal end portion; and
an attachment structure for removably attaching the housing to the patient's body.

17. The system of claim 16, wherein the spring member is a coil spring mounted between a first stationary cleat defined on the base and the slider member, and the loop is fitted around the slider, a second fixed cleat and a third fixed cleat.

18. The system of claim 16, wherein the attachment structure comprises an adhesive layer attached to a bottom exterior surface of the base.
